(19) United States
(12) Patent Application Publication
(10) Pub. No.: US 2026/0207697 A1
(43) Pub. Date: Jul. 23, 2026

(54) COMPOSITION FOR PREVENTION OR TREATMENT OF DISEASES RELATED TO COGNITIVE IMPAIRMENT OR MEMORY DISORDER, COMPRISING LIGUSTRUM FOLIOSUM EXTRACT

(71) Applicant: KOREA INSTITUTE OF ORIENTAL MEDICINE, Daejeon (KR)

(72) Inventors: Malk Eun PAK, Daegu (KR); Hye Jin YANG, Daejeon (KR); Younghoon GO, Gyeongsan-si (KR); Buyun KIM, Daegu (KR); Jae Kwang KIM, Daegu (KR); Jang-Gi CHOI, Daegu (KR); Wei LI, Seoul (KR)

(73) Assignee: KOREA INSTITUTE OF ORIENTAL MEDICINE, Daejeon (KR)

(21) Appl. No.: 19/142,288

(22) PCT Filed: Oct. 18, 2023
(86) PCT No.: PCT/KR2023/016098

§ 371 Date: Jun. 23, 2025

(30) Foreign Application Priority Data
Dec. 23, 2022 (KR) 10-2022-0183104
Jun. 21, 2023 (KR) 10-2023-0079714

(51) Int. Cl. A61K 36/638; A61P 25/28

(52) U.S. Cl. CPC A61K 36/638; A61P 25/28; A61K 2236/33

(57) ABSTRACT

The present invention relates to a composition for the prevention, improvement, or treatment of diseases related to cognitive impairment or memory disorder, comprising Ligustrum foliosum extract.

The Ligustrum foliosum extract according to the present invention exhibits effects of protecting neuronal cells, inhibiting cell death, and improving cognitive function, and thus can be usefully used as a therapeutic agent for diseases related to cognitive impairment or memory disorder.

### TECHNICAL FIELD

The present invention relates to a composition for the prevention, improvement, or treatment of diseases related to cognitive impairment or memory disorder, comprising Ligustrum foliosum extract.

### BACKGROUND ART

In modern society, where life is becoming more sophisticated due to the advancement of science and life expectancy is increasing due to the advancement of medicine, cognitive function and memory ability are of great interest to everyone, from adolescents with a large learning load to the elderly.

Cognitive ability encompasses memory, visuospatial perception, judgment, language, and calculation abilities, and allows one to perform daily life tasks without the help of others. However, in all cases of brain damage, such as rapid neuronal cell death caused by stroke, trauma and the like, or slow neuronal cell death causing degenerative brain diseases, irreversible dysfunction of the neural circuitry occurs immediately. Eventually, cognitive abilities decline, leading to diseases such as forgetfulness, memory disorder, dementia, and Alzheimer's disease, and making it impossible to maintain the same level of daily life as before. The number of patients with cognitive impairment, which causes not only personal suffering but also a decline in the productive population, is expected to increase to more than 74.7 million by 2030 (Abate G, 2017, Oxid Med Cell Longev. 2017:10), and the development of therapeutic agents or treatment methods for cognitive impairment has emerged as an urgent issue.

Memory loss causes problems in modern social activities and can make social life impossible when memory loss develops into a degenerative disease such as dementia, and the importance of studies on memory loss is increasing day by day.

To date, a large number of patents related to compositions that have the efficacy of treating cognitive impairment, improving cognitive ability, or enhancing memory ability have been published and registered in Korea, and representative published patents are as follows. Publication No. 2014-0144785 relates to a composition for the enhancement of memory and learning abilities comprising yuzu extract as an active ingredient; Registration No. 10-1823892 relates to Impatiens balsamina extract that exhibits effects of improving memory ability, improving cognitive ability, and preventing, delaying, or treating dementia. However, despite the availability of these chemical agents and natural compositions on the market, excellent preparations with proven efficacy have not yet been developed. Additionally, commercially available drugs cause a number of side effects, including hepatotoxicity, insomnia, high blood pressure, and vomiting. Therefore, there is an urgent need to develop a drug that can effectively treat cognitive impairment or memory disorder with few side effects.

### DISCLOSURE

### Technical Problem

The present inventors have conducted extensive studies to develop a substance that can prevent or treat diseases related to cognitive impairment or memory disorder, and as a result, have found that Ligustrum foliosum extract exhibits an excellent effect of improving cognitive function and memory ability, thereby completing the present invention.

### Technical Solution

An object of the present invention is to provide a pharmaceutical composition for the prevention or treatment of a disease related to cognitive impairment or memory disorder, comprising Ligustrum foliosum extract as an active ingredient.

Another object of the present invention is to provide a food composition for the prevention or improvement of a disease related to cognitive impairment or memory disorder, comprising Ligustrum foliosum extract as an active ingredient.

Still another object of the present invention is to provide a feed composition for the prevention or improvement of a disease related to cognitive impairment or memory disorder, comprising Ligustrum foliosum extract as an active ingredient.

Still another object of the present invention is to provide a method for preventing or treating a disease related to cognitive impairment or memory disorder, the method including administering a composition comprising Ligustrum foliosum extract as an active ingredient to a subject.

Still another object of the present invention is to provide a use of a composition comprising Ligustrum foliosum extract as an active ingredient for the prevention, treatment, or improvement of cognitive impairment or memory disorder.

### Advantageous Effects

The Ligustrum foliosum extract according to the present invention exhibits effects of protecting neuronal cells, inhibiting cell death, and improving cognitive function, and thus can be usefully used as a therapeutic agent for diseases related to cognitive impairment or memory disorder.

### BRIEF DESCRIPTION OF DRAWINGS

FIG. 1 is a graph illustrating an effect of protecting neuronal cells from oxidative stress induced by glutamate of Ligustrum foliosum extract;

FIG. 2 is a graph illustrating effects of protecting neuronal cells from oxidative stress induced by glutamate of extracts from different parts of Ligustrum foliosum;

FIG. 3 is a graph comparing effects of protecting neuronal cells from oxidative stress induced by glutamate of extracts of Ligustrum foliosum, Ligustrum japonicum, and Ligustrum obtusifolium;

FIG. 4 is a graph illustrating an effect of protecting neuronal cells from cell death induced by amyloid beta of Ligustrum foliosum extract;

FIG. 5 illustrates a schematic diagram of an animal experiment (in vivo);

FIG. 6 is a graph illustrating the results of a novel object recognition test (NORT) using an Alzheimer's mouse model;

FIG. 7 is a graph illustrating the results of a Y-maze test using an Alzheimer's mouse model;

FIG. 8 illustrates a schematic diagram of a scopolamine-induced cognitive decline animal experiment (in vivo); and FIG. 9 is a graph illustrating the results of a Y-maze test using a scopolamine-induced cognitive decline animal model.

### DETAILED DESCRIPTION OF THE INVENTION

This is specifically explained as follows. Meanwhile, each description and each embodiment disclosed in the present invention may also be applied to other descriptions and other embodiments, respectively. In other words, all combinations of the various elements disclosed in the present invention fall within the scope of the present invention. Additionally, the scope of the present invention should not be considered limited by the specific description described below.

Furthermore, those skilled in the art will recognize, or be able to ascertain using no more than routine experimentation, many equivalents to the specific embodiments of the present invention described herein. Additionally, such equivalents are intended to be included in the present invention.

An aspect of the present invention provides a pharmaceutical composition for prevention or treatment of a disease related to cognitive impairment or memory disorder, comprising Ligustrum foliosum extract as an active ingredient.

The term “Ligustrum foliosum” of the present invention belongs to the genus Ligustrum of the family Oleaceae, and is a deciduous broad-leaved tree that grows in sunny areas of mountains or fields. Ligustrum foliosum is a Korean endemic species distributed in Ulleungdo. The height thereof is approximately 1 to 3 m, and young branches thereof may or may not have fine hairs. The leaves are opposite, egg-shaped, ovate or elliptic, with smooth margins and fine hairs on the veins on the back. It has not been known that Ligustrum foliosum is used for the prevention and treatment of forgetfulness or memory disorder or the enhancement of memory ability, cognitive function, and learning ability, and the use of Ligustrum foliosum has been first found by the present inventors.

In the present invention, the plant may refer to a specific part, such as the root, leaf, flower, or stem of Ligustrum foliosum, or may refer to the entire plant. In a specific embodiment, the composition of the present invention may contain, but is not limited to, the leaf extract of Ligustrum foliosum.

As Ligustrum foliosum in the present invention, a commercially available one may be purchased and used, or one that is collected or grown in nature may be used, but Ligustrum foliosum is not limited thereto.

The term “extract” of the present invention refers to a result, such as a liquid component obtained by immersing a target substance in various solvents and then subjecting the target substance to extraction for a certain period of time at room temperature or in a heated state, or a solid component obtained by removing the solvent from the liquid component. The extract may be interpreted comprehensively to include, in addition to the result, a diluted liquid of the result, a concentrated liquid of these, an adjusted product of these, a purified product of these, and the like. Accordingly, the Ligustrum foliosum extract provided in the present invention may be interpreted as including the extract itself and all formulations of the extract that can be formed using the extract, such as an extract obtained by subjecting Ligustrum foliosum to extraction, a diluted or concentrated liquid of the extract, a dried product obtained by drying the extract, an adjusted or purified product of the extract, or a mixture of these.

In the Ligustrum foliosum extract of the present invention, the method for extracting the mixture is not particularly limited, and extraction may be performed according to a method commonly used in the art. Non-limiting examples of the extraction method include hot water extraction, ultrasonic extraction, filtration, and reflux extraction, and these may be performed singly or in combination of two or more methods.

In the present invention, the kind of solvent used for the extraction is not particularly limited, and any solvent known in the art may be used. The Ligustrum foliosum extract may be obtained by subjecting Ligustrum foliosum or its dried product, processed product, or the like to extraction singly or in combination using water or various organic solvents. At this time, the organic solvent used is not particularly limited, but may be, as an example, a polar solvent or a nonpolar solvent, may be, as another example, water, spirits, a lower alcohol having 1 to 4 carbon atoms (such as methanol, ethanol, propanol, or butanol), and mixed solvents thereof, and may be, as still another example, a mixed solvent of water and spirits, but is not limited thereto.

The term “cognitive function” of the present invention refers to various intellectual abilities such as attention ability, perception ability, memory ability, language ability, and executive ability. In the present invention, cognitive function may be used interchangeably with “cognitive ability”, “cognitive capability” or the like.

In the present invention, the term “cognitive impairment” is a concept that may include a state caused by a decline in cognitive function as above, a disease resulting therefrom, and all diseases that have such a state as a symptom, and preferably may mean a disorder caused by damage to brain neuronal cells.

The term “memory” or “memory ability” used in the present invention refers to the ability to acquire new information, learned experiences, or knowledge obtained from the surrounding environment, encode and store these in a specific part of the brain, and recall these.

The term “memory disorder” used in the present invention refers to a disease in which the ability to retain and recognize information, experiences, and stimuli is lost or impaired, and includes hypermnesia or memory decline. Memory disorder may occur if any one of the three processes of memory recording, maintenance, and retrieval is impaired.

In the present invention, the disease related to cognitive impairment or memory disorder may specifically be a degenerative brain nervous system disease; more specifically, the disease related to cognitive impairment or memory disorder may refer to a degenerative brain disease selected from the group consisting of mild cognitive impairment, Alzheimer's dementia, Parkinson's disease, amnesia, dementia, Pick's disease, and Huntington's disease.

In the present invention, “prevention” means any action of suppressing cognitive impairment or memory disorder or delaying the onset thereof by administering the pharmaceutical composition according to the present invention, and “treatment” means any action of improving or beneficially changing symptoms of cognitive impairment or memory disorder by administering the pharmaceutical composition.

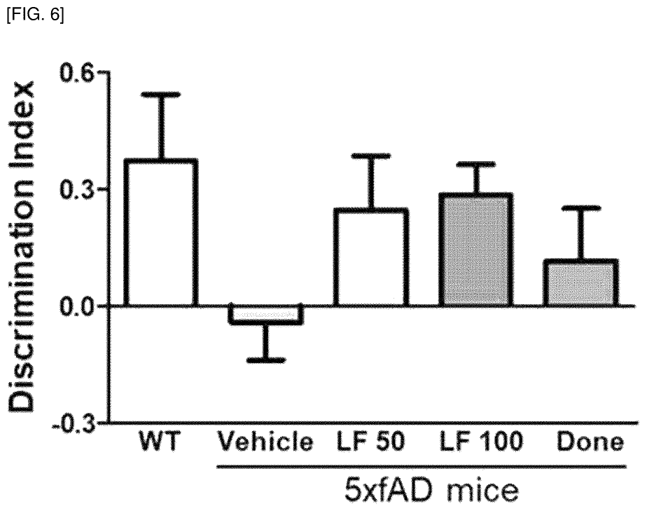

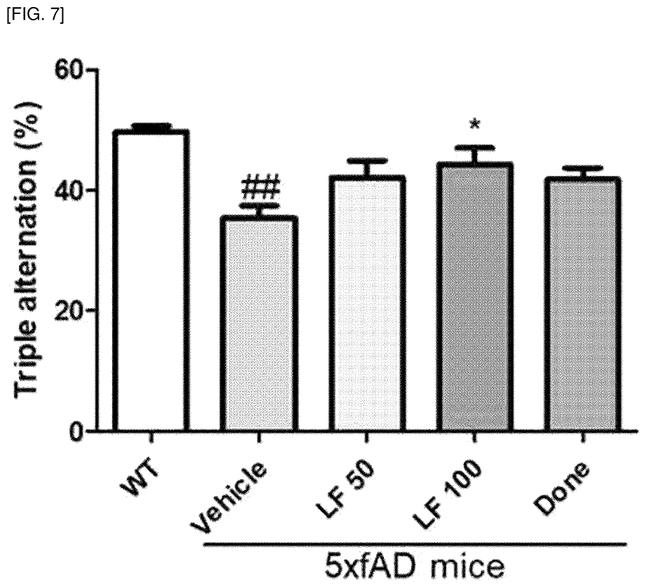

In a specific example of the present invention, the cognitive ability and memory ability improving effect of Ligustrum foliosum extract has been examined by constructing an Alzheimer's disease model and conducting a novel object recognition test and a Y-maze test, and as a result, it has been found that the cognitive ability and memory ability enhancing effect is superior to that in a case where the Ligustrum foliosum extract is not administered (FIGS. 6 and 7).

From this, it can be seen that the composition of the present invention comprising Ligustrum foliosum extract can be usefully used for the prevention or treatment of diseases related to cognitive impairment or memory disorder, and further for the improvement of cognitive function, treatment of cognitive decline, or improvement of memory ability.

The composition of the present invention may protect neuronal cells from oxidative stress induced by glutamate.

The term “glutamate” of the present invention is a major neurotransmitter in the central nervous system (CNS), but causes neurocytotoxicity and cell death at high concentrations. In hippocampal cells, glutamate reduces the intracellular antioxidant molecule glutathione, inhibits the uptake of N-acetyl-cysteine, and causes oxidative stress and cell death. It is also said that glutamate is involved in the development of some neurological disorders, including stroke and ischemia.

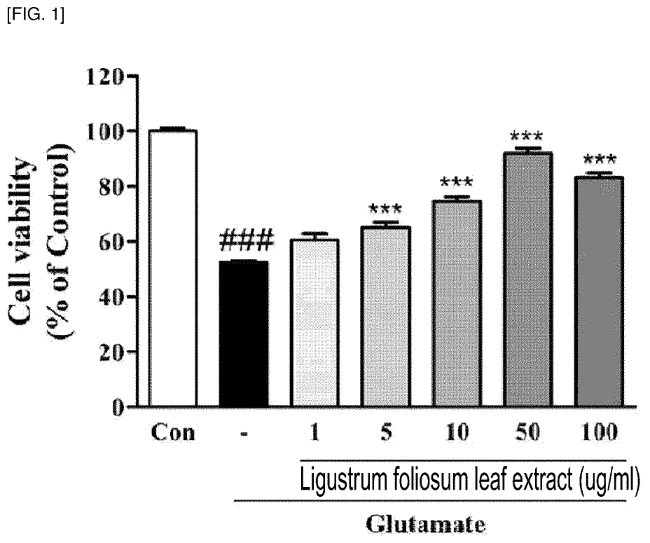

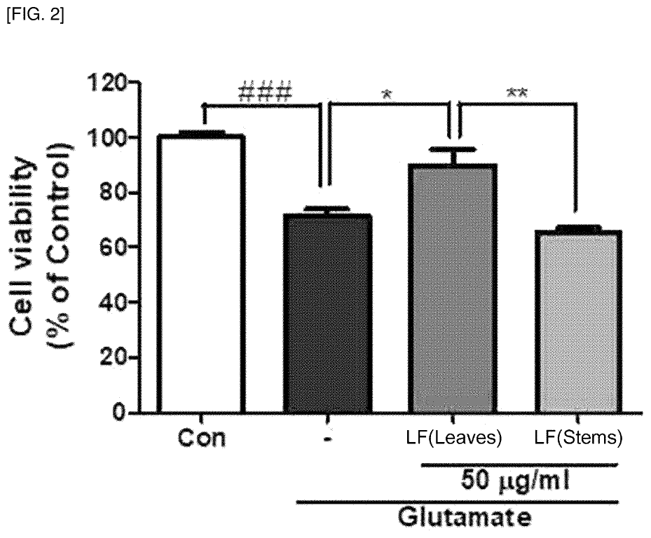

In a specific example of the present invention, it has been found that the survival rate of damaged neuronal cells increases when the neuroanl cells are treated with Ligustrum foliosum extract and glutamate (FIG. 1). In particular, it has been found that the Ligustrum foliosum leaf extract significantly increases the survival rate of damaged neuronal cells compared to the Ligustrum foliosum stem extract (FIG. 2).

From this, it can be seen that the composition comprising Ligustrum foliosum extract of the present invention has an effect of protecting neuronal cells from oxidative stress and an effect of inhibiting glutamate toxicity, and thus has an excellent effect of preventing or treating diseases related to cognitive impairment or memory disorder.

The composition of the present invention may protect neuronal cells from cell death induced by amyloid protein (amyloid β, Aβ).

The term “amyloid protein” of the present invention refers to a peptide composed of 36 to 43 amino acids, which is a major component of amyloid plaques found in the brains of Alzheimer's patients and is critically involved in Alzheimer's disease. It is known that neuronal cells die due to extracellular beta-amyloid deposition and intracellular hyperphosphorylated tau protein throughout the brain.

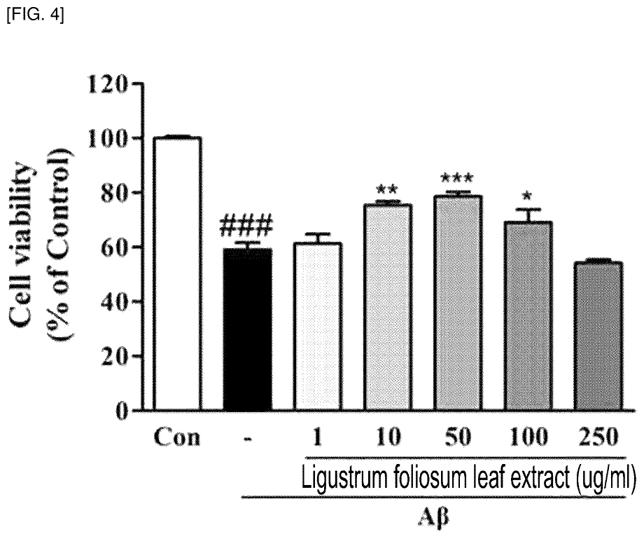

In a specific example of the present invention, it has been found that the survival rate of damaged neuronal cells increases through inhibition of cell death induced by amyloid protein when the neuronal cells are treated with Ligustrum foliosum extract and amyloid protein (FIG. 4).

From this, it can be seen that the composition comprising Ligustrum foliosum extract of the present invention has an effect of protecting neuronal cells from amyloid protein, and thus has an excellent effect of preventing or treating diseases related to cognitive impairment or memory disorder.

The composition of the present invention may have an effect of improving cognitive function from cognitive decline induced by scopolamine.

The “scopolamine” of the present invention is a competitive antagonist that blocks muscarinic receptors (mAChR), one of the subtypes of cholinergic neurons, and prevents acetylcholine from binding to the receptor in the synaptic gap without changing the amount of acetylcholine, which results in memory decline similar to the phenomenon caused by damage to cholinergic neurons in the central nervous system.

In an example of the present invention, it has been found that the effect of improving spatial perception and cognitive function increases compared to the control group when the neuronal cells are treated with Ligustrum foliosum extract and scopolamine (FIG. 9).

From this, it can be seen that the composition comprising Ligustrum foliosum extract of the present invention has an effect of improving memory loss and enhancing cognitive ability, and thus has an excellent effect of preventing or treating diseases related to cognitive impairment or memory disorder.

The “pharmaceutical composition” of the present invention may additionally contain a pharmaceutically acceptable carrier, excipient or diluent commonly used in the manufacture of pharmaceutical compositions. Such a pharmaceutically acceptable carrier, excipient, or diluent may be non-naturally occurring. Specifically, the composition may be used by being formulated in the form of oral formulations, such as powders, granules, tablets, liquids, capsules, suspensions, emulsions, syrups, and aerosols, external preparations, suppositories, and sterile injectable solutions, each according to a conventional method. In the present invention, carriers, excipients and diluents that may be contained in the pharmaceutical composition include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil. When the composition is prepared into a preparation, the preparation is usually prepared using diluents or excipients such as fillers, bulking agents, binders, wetting agents, disintegrants, and surfactants. Solid preparations for oral administration include tablets, pills, powders, granules, capsules, and the like, and these solid preparations are prepared by mixing the extract and its fractions with one or more excipients, for example, starch, calcium carbonate, sucrose or lactose, and gelatin. In addition to simple excipients, lubricants such as magnesium stearate and talc are also used. Liquid preparations for oral administration include suspensions, oral solutions, emulsions, and syrups, and in addition to the commonly used simple diluents such as water and liquid paraffin, various excipients, for example, wetting agents, sweeteners, flavoring agents, and preservatives may be contained. Preparations for parenteral administration include sterile aqueous solutions, non-aqueous solutions, suspensions, emulsions, lyophilized preparations, and suppositories. As non-aqueous solvents and suspending agents, propylene glycol, polyethylene glycol, vegetable oils such as olive oil, and injectable esters such as ethyl oleate may be used. As bases for suppositories, witepsol, macrogol, Tween 61, cocoa butter, laurin butter, and glycerogelatin may be used.

The content of the Ligustrum foliosum extract contained in the pharmaceutical composition of the present invention is not limited as long as the pharmaceutical composition has an effect of preventing or treating diseases related to cognitive impairment or memory disorder, but the Ligustrum foliosum extract may be contained at a content of 0.0001 to 99.9 wt %, more specifically 0.01 to 80 wt % based on the total weight of the final composition.

The pharmaceutical composition of the present invention may be administered in a pharmaceutically effective amount, and the term “pharmaceutically effective amount” of the present invention means an amount sufficient to treat or prevent a disease at a reasonable benefit/risk ratio applicable to medical treatment or prevention. The effective dose level may be determined depending on factors, including the severity of the disease, activity of the drug, age, weight, health, and sex of the patient, sensitivity of the patient to the drug, administration time, route of administration and excretion rate of the composition of the present invention used, treatment period, and drugs used in combination or concurrently with the composition of the present invention, as well as other factors well known in the medical field. The pharmaceutical composition of the present invention may be administered as an individual therapeutic agent or in combination with another therapeutic agent, and may be administered sequentially or simultaneously with conventional therapeutic agents. The pharmaceutical composition of the present invention may be administered in single or multiple doses. It is important to administer the pharmaceutical composition of the present invention in an amount that can achieve the maximum effect with the minimum amount without side effects, taking all of the factors into consideration.

As the dosage of the pharmaceutical composition of the present invention, for example, the pharmaceutical composition of the present invention may be administered to an animal, including a human, at 0.1 to 500 mg/kg of body weight per day, but is not limited thereto. The frequency of administration of the composition of the present invention is not particularly limited, but the composition may be administered once a day or several times a day in divided doses. The dosage does not limit the scope of the present invention in any way.

Another aspect of the present invention provides a food composition for the prevention or improvement of a disease related to cognitive impairment or memory disorder, comprising Ligustrum foliosum extract as an active ingredient.

The “Ligustrum foliosum”, “extract”, “cognitive impairment”, “memory disorder”, and “prevention” are as described above.

The term “improvement” of the present invention may mean any action of improving or beneficially changing symptoms of a disease related to cognitive impairment or memory disorder by administering the composition comprising Ligustrum foliosum extract of the present invention to a subject.

The term “food” of the present invention includes meat, sausage, bread, chocolate, candy, snacks, confectionery, pizza, ramen, other noodles, gum, dairy products including ice cream, various soups, beverages, tea, drinks, alcoholic beverages, vitamin complexes, health functional foods, and health foods, and includes all foods in the conventional sense. The food composition of the present invention can be consumed on a daily basis, thus can be expected to have a high effect of improving cognitive impairment or memory disorder, and can be greatly usefully used for health promotion purposes.

The health functional food is the same term as food for special health use (FoSHU), and refers to a food with high medical and healthcare effects that is processed to efficiently exhibit bioregulatory functions in addition to nutrition supply. Here, ‘functionality’ means obtaining a useful effect for health purposes, such as regulating nutrients for the structure and function of the human body or physiological actions. The food of the present invention can be manufactured by a method commonly used in the art, and may be manufactured by adding raw materials and ingredients commonly added in the art during the manufacturing process. In addition, the food of the present invention may be manufactured into any formulation without limitation as long as the formulation is recognized as food. The food composition of the present invention may be manufactured into various forms of formulations, has the advantage of not having side effects that may occur when drugs are taken for a long period of time as food is used as a raw material unlike general drugs, and is highly portable, therefore, the food of the present invention can be consumed as a supplement to enhance the effect of improving diseases related to cognitive impairment or memory disorder.

The health food refers to food that has a more active health maintenance or promotion effect than general food, and health supplement food refers to food for the purpose of health supplementation. In some cases, the terms health functional food, health food, and health supplement food are used interchangeably.

Specifically, the health functional food is a food manufactured by adding the compound of the present invention to food materials, such as beverages, tea, spices, gum, and confectionery, or by encapsulating, powdering, suspending, or the like the compound, and means to have a specific health effect when consumed, but has the advantage of not having the side effects that may occur when drugs are taken for a long period of time, as food is used as a raw material unlike general drugs.

The food composition may additionally contain a physiologically acceptable carrier. The kind of carrier is not particularly limited, and any carrier commonly used in the art may be used.

The food composition may also contain additional ingredients commonly used in food compositions to improve odor, taste, visuality, and the like. For example, vitamins A, C, D, E, B1, B2, B6, and B12, niacin, biotin, folate, and pantothenic acid may be contained. Additionally, minerals such as zinc (Zn), iron (Fe), calcium (Ca), chromium (Cr), magnesium (Mg), manganese (Mn), copper (Cu), and chromium (Cr); and amino acids, such as lysine, tryptophan, cysteine, and valine, may be contained.

The food composition may contain food additives, such as preservatives (potassium sorbate, sodium benzoate, salicylic acid, sodium dehydroacetate, and the like), sterilizers (bleaching powder and high-purity bleaching powder, sodium hypochlorite, and the like), antioxidants (butylated hydroxyanisole (BHA), butylated hydroxytoluene (BHT), and the like), coloring agents (tar pigments, and the like), color formers (sodium nitrite, sodium nitrite, and the like), bleaching agents (sodium sulfite), seasonings (monosodium glutamate (MSG), and the like), sweeteners (dulcin, cyclamate, saccharin, sodium, and the like), flavorings (vanillin, lactones, and the like), leavening agents (alum, D-potassium hydrogen tartrate, and the like), fortifying agents, emulsifiers, thickeners, film-forming agents, gum-forming agents, foam suppressants, solvents, and improvers. The additives may be selected depending on the kind of food and used in an appropriate amount.

As an example, the food composition of the present invention may be used as a health beverage composition, and in this case, may contain various flavoring agents or natural carbohydrates and the like as additional ingredients, like a regular beverage. The natural carbohydrates mentioned above may be monosaccharides such as glucose and fructose; disaccharides such as maltose and sucrose; polysaccharides such as dextrin and cyclodextrin; and sugar alcohols such as xylitol, sorbitol and erythritol. As sweeteners, natural sweeteners such as thaumatin and stevia extract; synthetic sweeteners such as saccharin and aspartame; and the like may be used. The proportion of the natural carbohydrates may be generally about 0.01 to 0.04 g, specifically about 0.02 to 0.03 g per 100 mL of the health beverage composition of the present invention. In addition to the above, the health beverage composition may contain various nutrients, vitamins, electrolytes, flavoring agents, coloring agents, pectic acid, salts of pectic acid, alginic acid, salts of alginic acid, organic acids, protective colloid thickeners, pH adjusters, stabilizers, preservatives, glycerin, alcohols, or carbonating agents. The health beverage composition may also contain fruit pulp for the production of natural fruit juice, fruit juice beverage, or vegetable beverage. These ingredients may be used independently or in mixture. The proportion of these additives is not greatly important, but is generally selected in the range of 0.01 to 0.1 parts by weight per 100 parts by weight of the health beverage composition of the present invention.

Still another aspect of the present invention provides a feed composition for the prevention or improvement of a disease related to cognitive impairment or memory disorder, comprising Ligustrum foliosum extract as an active ingredient.

The “Ligustrum foliosum”, “extract”, “cognitive impairment”, “memory disorder”, “prevention”, and “improvement” are as described above.

The term “feed” in the present invention means any natural or artificial diet, meal or the like or a component of the meal, which is intended for eating, ingesting, and digesting by an animal or suitable therefor.

The kind of feed is not particularly limited, and feed commonly used in the art may be used. Non-limiting examples of the feed include plant-based feed such as grains, roots, food processing by-products, marine algae, fiber, pharmaceutical by-products, oils, starches, cucurbit, or grain by-products; and animal-based feed such as proteins, inorganic substances, oils, minerals, oils, single-cell proteins, zooplankton, or food. These may be used singly or in a mixture of two or more kinds thereof.

Still another aspect of the present invention provides a method for preventing or treating a disease related to cognitive impairment or memory disorder, the method including administering a composition comprising Ligustrum foliosum extract as an active ingredient to a subject.

The “Ligustrum foliosum”, “extract”, “cognitive impairment”, “memory disorder”, “prevention”, and “treatment” are as described above.

The term “subject” as used in the present invention may mean any animal, including humans, that has or is likely to have cognitive impairment or memory disorder. The animal may be, but is not limited to, humans as well as mammals, such as cows, horses, sheep, pigs, goats, camels, antelopes, dogs, and cats, that require treatment for symptoms similar to those of humans.

The preventive or therapeutic method of the present invention may specifically include administering the composition in a pharmaceutically effective amount to a subject having or at risk of developing cognitive impairment or memory disorder.

The term “administration” as used in the present invention means introducing the pharmaceutical composition of the present invention to a patient by any appropriate method. As the administration route of the composition of the present invention, the composition may be administered via various routes such as an oral or parenteral route as long as the composition can reach the target tissue.

Still another aspect of the present invention provides a use of a composition comprising Ligustrum foliosum extract as an active ingredient for the prevention, treatment, or improvement of cognitive impairment or memory disorder.

The “Ligustrum foliosum”, “extract”, “cognitive impairment”, “memory disorder”, “prevention”, “improvement”, and “treatment” are as described above.

The composition may be a pharmaceutical composition, a food composition or a feed composition.

### [Mode for Carrying out the Invention]

Hereinafter, the present invention will be described in more detail through Examples and Experimental Examples. However, these Examples and Experimental Examples are intended to exemplify the present invention, and the scope of the present invention is not limited to these Examples and Experimental Examples.

### EXAMPLE 1

### Preparation of Ligustrum foliosum Extract

Leaves of Ligustrum foliosum collected from Ulleungdo were dried and crushed, 60 g of the leaves were added to 1 L of 99.9% methyl alcohol (HPLC grade) and subjected to extraction at room temperature using an ultrasonic extractor for 30 cycles. Extraction was performed under the conditions of 40 KHz, 1500 W, 15 minutes ultrasonication-120 minutes per cycle. Thereafter, filtration and concentration under reduced pressure were performed to prepare the extract, and the yield was 13.68%. The extract was provided by the Natural Product Central Bank and used in the experiment.

### EXAMPLE 2

### Examination of Effect of Protecting Neuronal Cell from Oxidative Stress Induced by Glutamate

### EXAMPLE 2-1

### Examination of Neuronal Cell Protecting Effect of Ligustrum foliosum Extract

The experiment was conducted using a mouse hippocampal neuronal cell line (HT22 cell line) constructed by isolating hippocampal brain tissue, which is known to be responsible for cognitive function. Specifically, the cells were seeded onto a plate and stabilized for 24 hours. Thereafter, the cells were pretreated with the Ligustrum foliosum extract at concentrations of 1, 5, 10, 50, and 100 g/mL and cultured for 24 hours. Next, the cells were treated with 5 mM glutamate, which exhibits excitotoxicity, and after 24 hours, treated with MTT solution to examine the cell viability through measurement of formazan crystals in living cells (FIG. 1).

As a result, it has been found that the Ligustrum foliosum extract increases the survival rate of damaged neuronal cells at concentrations of 5 to 100 μg/mL.

Through this, it can be seen that the Ligustrum foliosum extract of the present invention has an excellent effect of protecting neuronal cells from oxidative stress induced by glutamate.

### EXAMPLE 2-2

### Comparison of Neuronal Cell Protecting Effects of Extracts from Different Parts of Ligustrum foliosum

In order to compare the effects of Ligustrum foliosum (LF) leaf and stem extracts on neurotoxicity induced by glutamate, an experiment was conducted by the same method as in Example 2-1 (FIG. 2).

As a result, it has been found that the Ligustrum foliosum leaf extract significantly increases the cell viability decreased by glutamate compared to the Ligustrum foliosum stem extract.

Through this, it can be seen that the Ligustrum foliosum leaf extract of the present invention has an excellent effect of inhibiting cell death and protecting neuronal cells from oxidative stress induced by glutamate compared to extracts from other parts of Ligustrum foliosum.

### EXAMPLE 2-3

### Comparison of Neuronal Cell Protecting Effects of Ligustrum foliosum Extract, Ligustrum japonicum Extract, and Ligustrum obtusifolium Extract

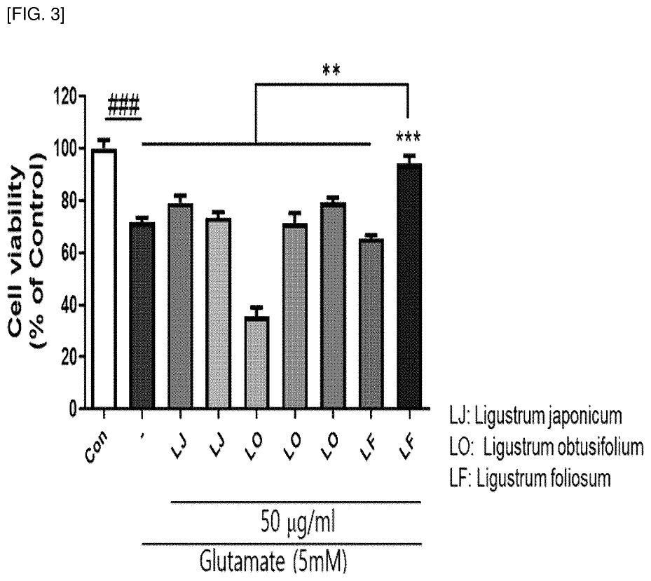

In order to compare the effects of Ligustrum japonicum (LJ) extract, Ligustrum obtusifolium (LO) extract, and Ligustrum foliosum (LF) extract on neurotoxicity induced by glutamate, an experiment was conducted by the same method as in Example 2-1 (FIG. 3).

As a result, it has been found that the Ligustrum foliosum extract significantly increases the cell viability decreased by glutamate compared to the Ligustrum japonicum (LJ) extract and the Ligustrum obtusifolium (LO) extract; in particular, it has been found that the Ligustrum foliosum leaf extract affords superior cell viability compared to the Ligustrum japonicum (LJ) leaf extract and the Ligustrum obtusifolium (LO) leaf extract.

Through this, it can be seen that the Ligustrum foliosum leaf extract of the present invention has an excellent effect of inhibiting cell death and protecting neuronal cells from oxidative stress induced by glutamate compared to other extracts.

### EXAMPLE 3

### Examination of Effect of Protecting Neuronal Cells from Cell Death Induced by Amyloid Protein (Amyloid β, Aβ)

The cells (HT22 cell line) were seeded onto a plate and then stabilized for 24 hours. Thereafter, the cells were pretreated with the Ligustrum foliosum extract at 1, 10, 50, 100, and 250 μg/mL for 4 hours, and then treated with 50 μM amyloid protein (Aβ 24 to 40) for neurotoxicity. After 48 hours, the MTT experiment was conducted to determine the cell viability (FIG. 4).

As a result, it has been found that the Ligustrum foliosum extract increases the survival rate of damaged neuronal cells at concentrations of 10 to 100 μg/mL.

Through this, it can be seen that the Ligustrum foliosum extract of the present invention has an excellent effect of inhibiting cell death induced by amyloid beta.

### EXAMPLE 4

### Construction of Alzheimer's Disease Model and Evaluation by Animal Experiment

### EXAMPLE 4-1

### Construction of Alzheimer's Disease Model and Drug Administration

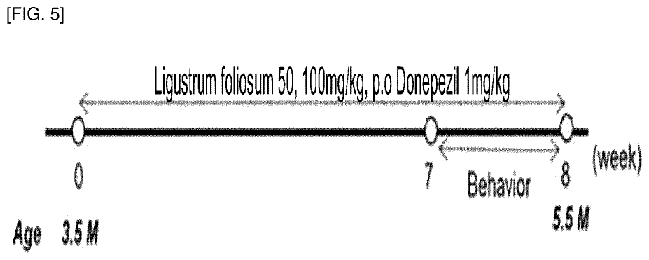

In order to examine the effect of treating cognitive impairment or memory disorder of Ligustrum foliosum extract, 5xfAD mice, which are mice transgenic for a gene associated with the development of Alzheimer's disease, were distributed, purchased and then bred for use in the experiment. The extract of the present invention or donepezil (positive control) dissolved in drinking water at 50 and 100 mg/kg or 1 mg/kg was orally administered to the distributed 14-week-old mice for 8 weeks, and drinking water (normal group and negative control group) was orally administered to the distributed 14-week-old mice for 8 weeks (FIG. 5).

### EXAMPLE 4-2

### Novel Object Recognition Test (NORT)

In order to test the memory ability of mice, the mice to which drugs had been administered in Example 4-1 were provided with two identical objects and allowed to explore the objects. The next day, one of the objects was replaced with a new object, and the time spent exploring each object again was measured (FIG. 6).

As a result, it has been found that the discrimination index of the control group (vehicle) significantly decreases compared to the normal group (Con, WT), and it has been found that the discrimination index significantly increases (recovered) when the mice are treated with the Ligustrum foliosum extract, and is significantly improved, particularly compared to the positive control group.

Through this, it can be seen that the Ligustrum foliosum extract of the present invention has the effect of enhancing cognitive ability and memory ability, and it can be seen that the Ligustrum foliosum extract has an effect of preventing or treating diseases related to cognitive impairment or memory disorder.

### EXAMPLE 4-3

### Y-maze Test

The Y-maze test is a behavioral experiment to determine the short-term memory of mice by calculating the ratio at which the mice select a new passage using an apparatus in which Y-shaped plastic passages are connected at an angle of 120° to each other. Spontaneous alternation is a measure of spatial working memory ability. Specifically, mice to which drugs were administered in Example 4-1 were placed in a Y-shaped maze and allowed to freely explore, and the pattern of exploration of each section was recorded. When the three passages were called passage A, passage B, and passage C respectively, a case where the mouse visited passages ABC, CAB, or BAC consecutively was considered as one alternation, and the alternation score was determined by measuring the ratio of the number of alternations to the total number of visits (FIG. 7).

As a result, it has been found that the alternation behavior of the control group (vehicle) significantly decreases compared to the normal group (Con, WT), and it has been found that the alternation behavior significantly increases when the mice are treated with the Ligustrum foliosum extract, and the alternation behavior is similar to or increases more than that of the positive control group particularly when the mice are treated with the extract of the present invention.

Through this, it can be seen that the Ligustrum foliosum extract of the present invention has an effect of improving the decline in spatial perception ability and short-term memory ability caused by diseases related to cognitive impairment, and it can be seen that the Ligustrum foliosum extract of the present invention has an effect of preventing or treating diseases related to cognitive impairment or memory disorder.

### EXAMPLE 5

### Construction of Scopolamine-Induced Cognitive Decline Animal Model and Evaluation by Animal Experiment

### EXAMPLE 5-1

### Construction of Scopolamine-Induced Cognitive Decline Animal Model and Drug Administration

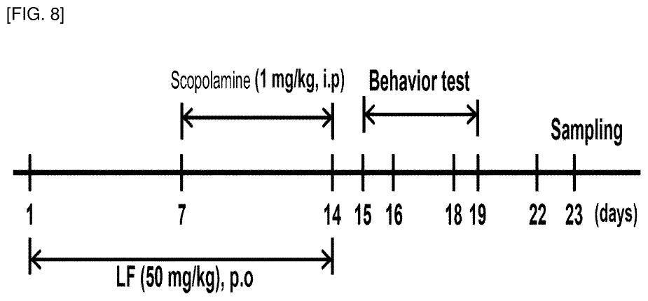

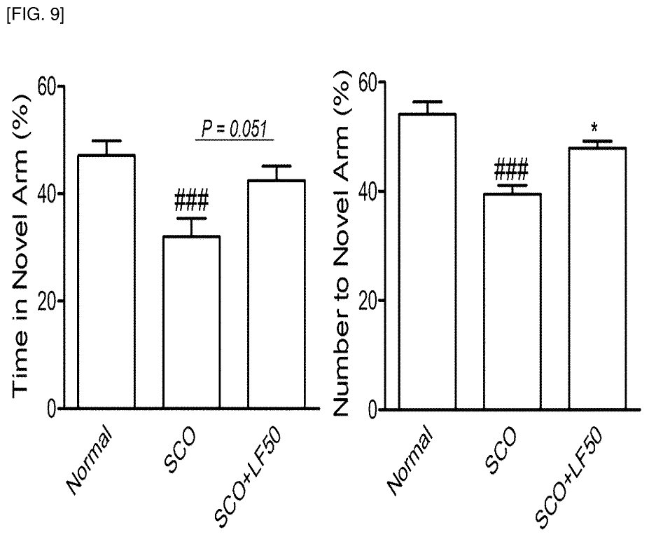

In order to examine the effect of treating cognitive impairment or memory disorder of the Ligustrum foliosum extract, a cognitive decline animal model (amnesia animal model) constructed by injecting scopolamine was used. Specifically, 7-week-old mice were purchased from Dooyeol Biotech and stabilized for one week for use in the experiment. After the Ligustrum foliosum extract dissolved in drinking water at 50 mg/kg was orally administered to the mice for 7 days, scopolamine was administered to the mice together with the leaf extract for 7 days. Drinking water and saline were administered to the normal group (Con) and drinking water and scopolamine were administered to the control group (SCO). The extract of the present invention or donepezil (positive control) group dissolved in drinking water at 50 and 100 mg/kg or 1 mg/kg was orally administered to the 8-week-old mice for 8 weeks, and drinking water (normal group and negative control group) was orally administered to the 8-week-old mice for 8 weeks (FIG. 8).

### EXAMPLE 5-2

### Y-Maze Test

The Y-maze test is an experiment to measure the spatial perception of mice by conducting two experiments, spaced two hours apart, using an apparatus in which Y-shaped plastic passages are connected at an angle of 120° to each other and calculating the ratio at which the mice select a new passage.

Specifically, one arm of the Y-shaped maze was closed, and mice to which drugs were administered in Example 5-1 were allowed to move freely and explore in the other two arms for 3 minutes (acquisition phase). After 2 hours, all arms were opened and the mice were allowed to move freely and explore for 3 minutes, and the number and time entered into the newly opened arm were recorded. Finally, the total number of times or final time entered into each arm was divided by the number of times or time entered into the newly opened arm, and the result was expressed as a percentage (FIG. 9).

As a result, it has been found that the time and number of times entered into the newly opened arm significantly decrease in the control group (vehicle) compared to the normal group (Con, WT), but it has been found that the time and number of times entered into the newly opened arm increase; in particular, the number of times entered into the newly opened arm significantly increases when the mice are treated with the Ligustrum foliosum extract.

Through this, it can be seen that the Ligustrum foliosum extract of the present invention has an effect of improving spatial perception in cognitive impairment induced by scopolamine and an effect of improving impaired cognitive function, and it can be seen that the Ligustrum foliosum extract of the present invention has an excellent effect of preventing or treating diseases related to cognitive impairment or memory disorder.

From the above description, those skilled in the art to which the present invention belongs will understand that the present invention can be implemented in other specific forms without changing the technical idea or essential characteristics thereof. In this regard, it should be understood that the Examples described above are exemplary in all respects and not restrictive. The scope of the present invention is defined by the appended claims rather than by the description preceding them, and therefore all changes or modifications derived from the meaning and scope of the patent claims and their equivalent concepts should be interpreted as being included in the scope of the present invention.

1. A for preventing or treating a disease related to cognitive impairment or memory disorder, including administering a composition comprising Ligustrum foliosum extract as an active ingredient.

2. The method of claim 1, wherein the Ligustrum foliosum extract is Ligustrum foliosum leaf extract.

3. The method of claim 1, wherein the disease related to cognitive impairment or memory disorder is a degenerative brain nervous system disease.

4. The method of claim 1, wherein the disease related to cognitive impairment or memory disorder is mild cognitive impairment, Alzheimer's dementia, or Parkinson's disease.

5. The method of claim 1, wherein the extract is extracted using water, a C1 to C4 lower alcohol, or a mixed solvent thereof.

6. The method of claim 1, wherein the composition protects neuronal cells from oxidative stress induced by glutamate.

7. The method of claim 1, wherein the composition protects neuronal cells from cell death induced by amyloid protein (amyloid β, Aβ).

8. The method of claim 1, wherein the composition has an effect of improving cognitive function from cognitive decline induced by scopolamine.

9.-14. (canceled)
